(19) United States
(12) Patent Application Publication
(10) Pub. No.: US 2008/0086325 A1
(43) Pub. Date: Apr. 10, 2008

(54) SYSTEM AND METHOD FOR MANAGING HEALTH RISKS

(76) Inventor: Terry L. James, Fairview, TX (US)

Correspondence Address: BAKER BOTTS L.L.P., 2001 ROSS AVENUE, SUITE 600, DALLAS, TX, 75201-2980, US

(21) Appl. No.: 11/538,598

(22) Filed: Oct. 4, 2006

(51) Int. Cl. G06Q 10/00; A61B 5/00

(52) U.S. Cl. 705/2; 600/300

(57) ABSTRACT

A system and method for managing health risks is provided. The system and method comprise identifying one or more relevant economic risk factors from health-related data collected from a person, providing an intervention plan to the person based on the relevant economic risk factors, authenticating an identity collected from a participant at a remote location, exchanging data related to the intervention plan with the person at the remote location, and providing an incentive to the person for complying with the intervention plan.

### TECHNICAL FIELD OF THE INVENTION

This invention relates in general to health management, and more particularly to a system and method for managing health risks.

### BACKGROUND OF THE INVENTION

Our nation currently spends over $1.5 billion on healthcare each year. The past twenty years has witnessed an unrelenting cost increases in healthcare. Just since 2002, costs have increased by thirty percent. Faced with an aging population and no end in sight to our ever-increasing healthcare expenditures, a myriad of potential solutions have been offered to slow, to reverse, or otherwise to reduce this problematic trend.

The proffered healthcare solutions have been many, including managed care, preferred provider organizations (PPOs), health maintenance organizations (HMOs), contracted services, plan designs, co-pay schemes, deductible strategies and consumer driven healthcare. These solutions initially seem diverse in appearance and unrelated in their approaches. They do, however, share common platforms. They focus on who is going to pay the incurred expenses (e.g. the employer versus the employee), how much providers of services (e.g. doctors and hospitals) are going to be paid, and how much the financial risk taker (e.g. insurance companies) will make for financing the uncertainty of who will experience illness and how much that illness will eventually cost. Engrained into this paradigm are suppliers and business support systems that offer their wares and services in hopes of participating in this ever-growing healthcare industry.

Employers often offer to share healthcare expenses with employees as a benefit to the employees. In such an arrangement, either the employer or the employee ultimately pays for the healthcare expenses. Once the employer offers healthcare as a benefit to employees, the employer assumes the risk of paying at least some portion of future healthcare expenses for those employees. If the employee population is healthy and requires little or no medical services, the employer's cost will be minimal. If the employee population is not healthy, then the employer's cost could be unaffordable. The employer then may choose to shift some of the risk (and some of the cost) to an insurer, the employees, or both.

An employer generally may shift costs to employees through various schemes such as: plan design, deductibles, co-pays, coverage limits, medical savings plans, etc. All of these schemes are designed to define who is going to pay: the employer or the employee.

An insurer is a financier of medical services. An insurer is paid to assume the risk for healthcare expenses that an employer is unwilling or unable to assume. For example, suppose an employer has 500 employees and the cost of insurance for those employees is $6,000 per employee per year (or a total of $3,000,000). The premium payment of $3,000,000 is the total financial risk for the employer. If two unexpected pre-mature babies are born and the cost of care for each of those babies is $500,000 or $1,000,000 and the total healthcare expense balloons unexpectedly to $4,000,000, then the insurer loses $1,000,000.

The insurer only becomes a payer because the employer shifted some of the risk by paying the insurer to assume any expenses in excess of the premium payments. In reality, the insurer is using the employer's money and employee's money to pay the bills in hopes that there is some money left over at the end of the day.

An insurer generally is in business to make a profit. A significant profit source is the difference between the money it takes in (premiums) and the money it pays out (medical expenses). Payment for medical services becomes a primary cost. As a result, an insurer is highly motivated to limit the amount of money it pays to hospitals, doctors, or other healthcare providers. In order to limit monies paid out for medical services, insurers also have implemented a variety of plans, including PPOs, HMOs, capitation arrangements, contracted services, drug purchase agreements, and the like. The plans are multiple and diverse, and all designed to increase profits for the insurer by reducing cost (i.e. money paid for medical services).

Healthcare costs continue to rise, though, and that is a problem—a serious problem. Someone has to pay for medical services and there always seems to be someone who wants or needs those services. It is interesting that the prevalent thinking of the day has approached the problem of rising healthcare cost with solutions that focus on financing the risk associated with healthcare cost. The solutions are all centered on money. Who pays? Who is at risk to pay? Who gets paid what if this happens?

It seems strange to approach the problem of healthcare, people getting sick or not sick, with strategies around money. To date no one has found a disease caused by money or cured by money. People do not get infected with money, and money does not cause cancer. Health, or the lack of it, is about people. People get sick. People are healthy or unhealthy. Surprisingly little attention has been given to the individual's role in the rising cost of healthcare. The ‘money people’ are looking for ‘money solutions.’ After all, business is business. But without the need or the desire of individuals to seek medical services, the costs go down because demand for services goes down.

In fact, without people who become patients, healthcare ceases to exist. Unless someone is sick, hurt, or in pain, no health service is tendered. Without a patient, doctors and hospitals cease to exist. The impetus that drives the system for the healthcare players (i.e., physicians, hospitals, pharmaceutical manufacturers, suppliers, and insurers) is the irrefutable truth that there is a patient, one who is in need of care. Remove the patient from the equation and, rather suddenly, the healthcare players dissolve. Nothing disturbs a physician more than an empty waiting room, or a hospital administrator more than a barren surgery schedule.

It seems universally accepted by the healthcare players, and the thinking of the status quo, that the patient is merely someone who stands in need of care, who knew nothing of his illness, and who lacks any responsibility for his condition. The common thinking of the day continues that this unfortunate patient, due to circumstances beyond his control, just became ill. The healthcare players' interest is to make a product and to provide care for whoever needs it, but never eliminate the need for services, never reduce the demand. Ask a hospital administrator about wellness and the reply will likely be, “Why would I want a wellness program? I make a profit from sick people, not well people.”

The question arises: do patients just get sick or are they a causal agent in the risk for disease development? Could the patient, the passive participant in this disease by chance occurrence hypothesis actually be a fundamental driver of healthcare costs? If they are passive, are not playing an active role in the demand for medical services, and are only by-products of random misfortune, then any strategy that considers them is futile. If, on the other hand, the patient is a causal agent, then the chance to influence him must be fundamental in a risk management solution designed to affect healthcare expenditures.

It is our belief that the individual is a fundamental causal agent in the risk for disease development and a driving force for subsequent healthcare cost. Individual choices are critical to determining the likelihood of the occurrence of disease and the severity of the disease process. Furthermore, once a specific disease condition is present, how an individual relates to that condition serves as a primary driver in the severity of the disease process and its resulting cost of care.

Creating strategies that focus on the individual, in our opinion, can significantly alter the risk for disease development and further reduce healthcare cost. It is the individual, who has been neglected as a cost center in healthcare expenditures. Indeed, certain efficiencies may exist, that can be found, if individual choices are addressed. Such choices are vitally important because they put the patient at risk for disease development and generate corresponding healthcare expenditures, driving cost upwards, each and every year.

### SUMMARY OF THE INVENTION

From the foregoing, it may be appreciated that a need has arisen for an improved process for achieving superior management of healthcare costs. In accordance with an embodiment of the present invention, a system and a method for managing healthcare costs are provided that focus on individual demand or need for healthcare services and substantially eliminate or greatly reduce disadvantages and problems associated with conventional healthcare approaches, strategies, and instruments.

According to an embodiment of the present invention, a method for managing health risks is provided that comprises identifying one or more relevant economic risk factors from health-related data collected from a person, providing an intervention plan to the person based on the relevant economic risk factors, authenticating an identity collected from a participant at a remote location, exchanging data related to the intervention plan with the person at the remote location, and providing an incentive to the person for complying with the intervention plan.

In another embodiment of the invention, a system is provided for use in a health management program. The system comprises a user interface, a network interface, a tracking component, and a processing component. The user interface receives a user identity from a person, and collects data related to an intervention plan for reducing one or more relevant economic risk factors, which are identified from health-related data associated with the person. The tracking component tracks compliance with the intervention plan. The processing component calculates a credit to the user for compliance with the intervention plan.

Certain embodiments of the present invention may provide a number of technical advantages. For example, according to one embodiment of the present invention, an architecture and process are provided that are comprehensive in nature. Each component in the process operates within a structure or framework of an overall scheme to produce a synergistic effect. For example, the design of an intervention is a consequence of identifying critical pieces of data that form relevant economic risk factors. Similarly, implementing an incentive program fosters participation in an intervention, which was designed in a preceding step. As is evident, standing alone, each of these steps has value. But united together, they form a powerful tool in effecting changes in the targeted individual or group. The integration of these critical (and co-dependent) steps yields a significant reduction in healthcare costs.

Another technical advantage of the present invention is a result of its unique focus. The present invention centers on the individual, who is a primary determinant in generating healthcare expenditures. The identified relevant economic risk factors stem directly from clinical observations, character observations, or disease states or conditions of an individual or group of individuals. These relevant economic risk factors then are used as the basis for configuring modules, which specifically address targeted clinical or character observations or the disease states in the targeted population which are potentially modifiable. Considerable time and effort is expended in designing modules that should achieve the most beneficial results for the target individual or group, and thereby alleviate healthcare costs. A module may include virtually any action, exercise, task, or assignment that is tailored to the individual or group in order to affect behavior. Modules may influence individual choices for either: 1) a pre existing disease; or 2) a set of circumstances or factors that may lead to the development of some disease or demand for medical services. Modules may be constructed from a central theme that suggests that the individual is a casual agent in a disease management (or disease prevention) process. Thus, individual choices are significant in the context of the severity of a disease or the prevention of a potential future affliction and its subsequent cost of care.

Such a technique is based on the realization that healthcare expenditures have little to do with what people know or do not know. Healthcare expenditures have far more to do with the choices people make, the effects that those choices have on the risk for disease development, and/or the relationship between such choices and an existing disease process. There is not a lack of information that is available to an individual. There is a lack of skill and application of that information, however. Thus, many of the modules presented herein address those elements of choice, as they relate to what people do, how they think, how they feel, and what they believe. A key element is to design interventions that focus on the process of change. This stands in stark contrast with rudimentary models that, for example, attempt to financially squeeze physicians and/or hospitals such that their billing rates are decreased. Such a senseless healthcare strategy fails to consider individual choices, which are critical components in the risk for disease and for the cost of care.

Yet another technical advantage associated with the present architecture is that it allows for greater specificity in measuring the economic efficacy of behavioral, chemical, and environmental changes (in the context of an intervention) for any individual or group. Such a measurement method may collect data from three domains and use this information to determine the economic efficacy. The results of the intervention(s) may be readily tracked over any desired time period such that a tangible result is produced. The resultant can then be used to offer convincing and compelling validated data associated with the intervention and a reduction in healthcare costs for the target group. This resultant provides verifiable knowledge associated with cost expenditures for any entity seeking to review such economics.

Certain embodiments of the present invention may enjoy some, all, or none of these advantages. Other technical advantages may be readily apparent to one skilled in the art from the following figures, description, and claims.

### BRIEF DESCRIPTION OF THE DRAWINGS

For a more complete understanding of the present invention and its advantages, reference is now made to the following description, taken in conjunction with the accompanying drawings, in which:

FIG. 1A is a simplified flowchart illustrating a process and a method for managing healthcare expenditures that illustrates five basic steps;

FIG. 1B is a simplified set of Venn diagrams associated with information used in the method and process of FIG. 1A;

FIG. 2 is a block diagram of a system that includes a number of example operations to be completed during the process and the method for managing healthcare expenditures;

FIG. 3 is an example listing of health risk appraisal data;

FIG. 4 is a simplified schematic diagram of a number of example modules that may be completed as part of the process and the method for managing healthcare expenditures;

FIG. 5 is a simplified schematic diagram illustrating the interaction between the patient's completion of assigned modules and the ability to participate in a gaming opportunity;

FIG. 6 is a simplified graphical illustration that shows a potential result of an intervention for a given company;

FIG. 7 is a simplified block diagram of a data processing system for delivering and administering certain features of the invention; and

FIG. 8 is a flow diagram that illustrates one embodiment of an algorithm associated with an embodiment of a health station associated with the invention.

### DETAILED DESCRIPTION OF THE INVENTION

For purposes of teaching and discussion, it is useful to provide some overview as to the way in which the following invention operates. The following foundational information may be viewed as a basis from which the present invention may be properly explained. Such information is offered earnestly for purposes of explanation only and, accordingly, should not be construed in any way to limit the broad scope of the present invention and its potential applications.

Managing expenses is critical in any business environment. Much could be learned from the corporate titans of the 19th and 20th centuries. For example, Andrew Carnegie (founder of U.S. Steel) once noted: “find the cost and reduce it.” His relentless efforts to drive down costs and to undersell the competition made his steel mills the most modern in the world: the models for the entire industry. A person cannot lower expenses until a person has identified the cost. This ideology is so important that it needs to be repeated. In any business, whether it be automobiles, computers, or healthcare, in order to be truly successful—find the cost and reduce it.

Turning to a fellow titan of industry, John D. Rockefeller is remembered as one of the foremost capitalists in American history. John D. Rockefeller created the Standard Oil Corporation, which (at the time) was the largest business empire on earth. When Standard Oil was first organized, their primary product was kerosene—not gasoline. Rockefeller focused on nearly every aspect of the business in order to reduce costs. For example, instead of purchasing barrels for $2.50 each, Standard Oil made their own barrels for $1 each. No detail was too small for Rockefeller.

Rockefeller was also known to go down to the refinery and sweep up with a broom in such a nonchalant manner that the workers would continue their efforts without pausing or noticing him. He questioned every aspect of factory work and made suggestions on how things might be improved ever so slightly. In the barrel-making effort, he noticed forty soldered rivets were used to secure the wood components to their steel counterparts. One day he asked the welder why this was so. The welder responded that this was how he was trained. Rockefeller asked him to try thirty-eight. This was unsuccessful as the barrels would burst when filled. Rockefeller then suggested thirty-nine beads of solder; this time the barrels held. Fifty years later, Rockefeller would delight in sharing this tale of how he had saved a fortune in the refining process with just such little modifications. He might be said to be one of the fathers of efficient cost reduction. The lesson here? Rockefeller was successful because he was able to identify costs and to reduce the expenses associated with those costs.

Turning to yet another captain of industry, Edward H. Harriman was the biggest railroad mogul of the 20th century. His company, Union Pacific, was reincorporated in 1897 as the Union Pacific Railroad Company of Utah. Under the management of E. H. Harriman, the railroad was expanded and vastly improved. In 1901, Harriman added the Southern Pacific and the Central Pacific to his expanding railroad empire, and his spectacular attempt to control the Northern Pacific led to the formation of the Northern Securities Company, a huge rail monopoly that controlled transportation throughout the Northwest.

On one occasion, Harriman was walking along the tracks with an assistant. Looking at a track bolt, he turned to his assistant and asked, “Why does so much of the bolt protrude beyond the nut?” The assistant responded, “I don't really know, except that it is the size we've always used.” The assistant seemed confused by the question, as it seemed to be a trivial and meaningless detail. “Why should we use a bolt of such length that a part of it is utterly useless?” asked Harriman. The assistant responded, “Well, when you come right down to it, there is no reason.” The two continued walking along the track for a moment, then Harriman asked how many track bolts there were in a mile of track. He was informed that Union Pacific had thousands of miles of track, and there must be some fifty million track bolts in the present Union Pacific railroad system. Harriman thought to himself for a moment, “If we can shorten that bolt and, thereby, cut an ounce from every bolt we use, we could save fifty million ounces of iron.” This is no small detail; this is critical. Harriman's conclusion is reflected in the following response to his assistant, “Change your bolt standard!”

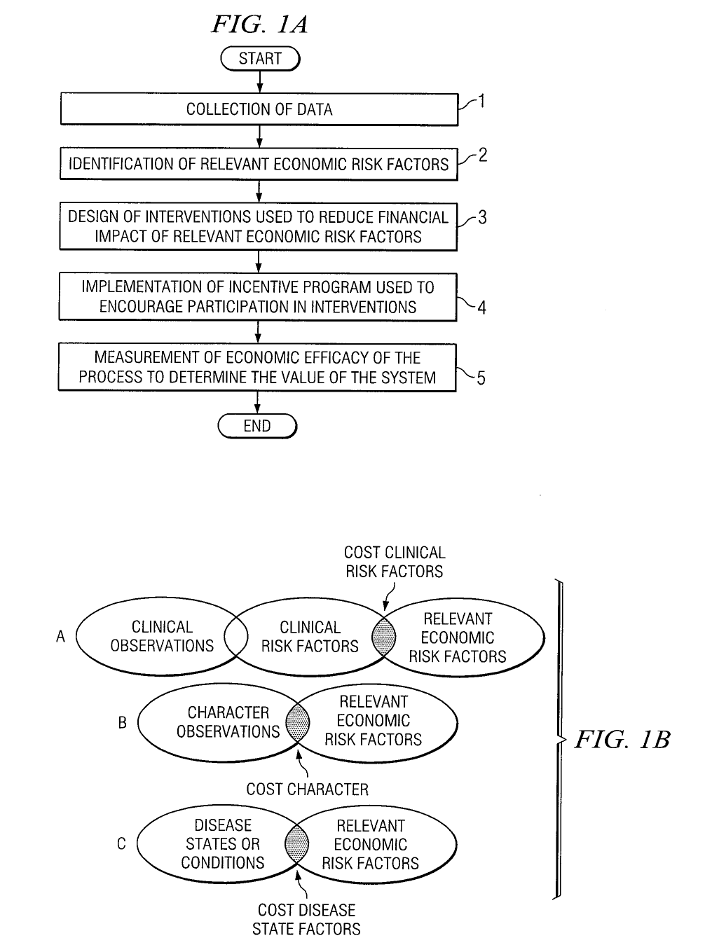

The important point to take from these accounts is that the most successful corporations and firms are those that figure out how to reduce costs. What did E. H. Harriman and John D. Rockefeller have in common? Succinctly stated, the commonality is the ability to accurately identify costs. Andrew Carnegie did it with steel, John D. Rockefeller with oil, E. H. Harriman with railroads, Henry Ford with cars, Michael Dell with computers, and Richard Sears and Sam Walton with retail stores. This cost-reducing theory is reflected in the healthcare management process of FIG. 1A. In the context of the previous examples, the flowchart of FIG. 1A illustrates identifying the cost (through Step 2) and reducing the expenses associated with the cost (through Steps 3 and 4). The steps of the process are outlined below in greater detail.

FIG. 1A is a simplified flowchart illustrating a process and a method that includes five basic steps to be completed in order to achieve effective management of health risks and associated expenses. FIG. 1A reflects a comprehensive process for the management of healthcare costs that focuses on the patient as a cost center and driver of healthcare expenses. The outlined process capitalizes on the fact that the demand for medical services can be modified by looking at the patient as a causal agent for the risk of disease development and the associated cost of care. This is in contrast to methods that merely focus on financial solutions for the rising healthcare costs as previously discussed. The illustrated process may be divided into five general steps, each of which is further detailed in subsequent FIGURES such that the audience is made aware of the extensive teachings of each step in the process. FIG. 1A is only offered to provide a broad framework from which to work—a framework that is fully supplemented by additional FIGURES and disclosure.

In step 1 of FIG. 1A, the target population (potentially a group of employees of Company Alpha) is introduced into the program, where data associated with the individuals is collected. In general, the data is health-related data, and this data collection step may be inclusive of gathering any information germane or pertinent to factors that contribute to a person's health. Step 2 involves the identification of relevant economic risk factors. These risk factors may serve as the basis for the step 3—the design and implementation of risk reduction interventions. In a similar comprehensive fashion, the step 4 provides an incentive system that gives traction to the interventions that were introduced into the target population. In a general sense, step 4 is a byproduct of the previous steps, as it is integrated into the process in order to give significance to step 3.

As is readily evident, the steps presented in FIG. 1A are interrelated and build on each other. These steps collectively generate an overall synthesis that offers a powerful strategy in attacking excessive healthcare expenditures. The final step (step 5) in the process measures the economic efficacy of the process such that the value of the entire system is ascertained. This last step offers a practical guide, as well as a tangible result, to any company official or director who seeks to identify real-world ramifications of such a unique approach. In contrast to existing disease management programs or ambiguous wellness proposals, which claim health cost savings by referencing literature and studies, step 5 is a direct measurement and identifies an actual bottom-line savings for any company that participates in such a process. The following description further explains these five steps in providing greater detail and examples associated with each component of the process.

FIG. 1B is a collection of three simplified Venn diagrams that may be used to clarify some of the terms used herein. Venn diagram A includes clinical observations, clinical risk factors, and relevant economic risk factors. A clinical risk factor is a clinical observation that has been statistically demonstrated to participate in the development of a given disease. For example, if a person is sedentary, obese, or is a smoker, the patient has clinical risk factors for heart disease. However, there are other clinical observations that would not qualify as a “clinical risk factor.” For example, the fact that the patient was a certain height or had poor vision would not necessarily qualify as a clinical risk factor for heart disease.

Similarly, not all clinical risk factors have economic value. Clinical risk factors tell you if someone is at risk for developing a disease or condition, but clinical risk factors do not tell you when that disease process is likely to occur and its potential cost for the party bearing the economic risk. In and of themselves, clinical risk factors lack economic value. A person with high blood pressure, high cholesterol, obesity, and a family history of heart disease is at risk to have a heart attack. But when will that heart attack take place? Next year? Or five years from now? Is there an economic threat to company Alpha next year due to these factors?

What is neglected in any discussion of clinical risk factors is timeliness—the economic value of a clinical risk factor next year or relevant time frame. By merging clinical risk factors with other data domains, the economic value of a clinical risk factor can be statistically determined. If a given clinical risk factor is determined to drive cost today and will continue to drive cost tomorrow it becomes a relevant economic risk factor and is called a cost clinical risk factor.

Relevant economic risk factors are economic drivers of healthcare expenses. They are cost determinants that have been found to have statistical relevance to current cost and predicting future costs within a selected time frame. Unless they are modified, relevant economic risk factors continue to push cost forward unabated. There are three kinds of relevant economic risk factors. One is a clinical risk factor, called a “cost clinical risk factor” as described beforehand. The second is called a “cost character.” The third is referred to as a “cost disease state factor.”

In order to determine the economic relevancy of a cost clinical risk factor, or cost character or cost disease state factor, it is important to obtain utilization data. Utilization data includes economic data related to healthcare expense for an individual or group. It includes hospitalization fees, drugs fees, doctor fees, laboratory fees, premiums for insurance, x-rays. If a charge or fee is associated with a given individual or group for any heath related expense or medically related event then that charge should be part of the utilization history. The nature of defining a relevant economic risk factor is the establishment of the relationship between clinical information (clinical risk factors, biometrics, personal history) and financial information (what is spent). It is the predictive nature of the relevant economic risk factor that permits the identification of cost drivers that are driving cost today and will continue to drive cost tomorrow.

Venn diagram B includes a group referred to as “character observations.” Character observations are non-clinical observations of an individual or a group. Whereas clinical observations refer to observations that may have clinical significance, character observations are observations representing characteristics of the individual or group that could lead to consistent generation of healthcare costs. If such a finding (when evaluated with other data domains) is found to have statistical relevance to drive cost over time it becomes a relevant economic risk factor and is called a cost character.

These terms may be understood better in the context of an example. Consider the case of the ‘hookworm’ crisis. Hookworm was a plague that reached epidemic proportions at the turn of the century. The disease was particularly a problem for the populations of the southern states in the U.S. Since many Southerners did not wear shoes in the summer months, hookworm larva often penetrated between people's toes. After making its way through the victim's respiratory tract, the larva eventually found their way to the small intestine about a week later. The disease progressed until the patient started exhibiting more dangerous symptoms. Hookworm can produce anemia, abdominal pain, diarrhea, loss of appetite, and weight loss. In worst cases, hookworm can cause stunted growth and problems with mental development.

Thus, the character observation of Venn diagram B would be the fact that certain people did not wear shoes. The character observation was also a cost character due to the fact that because certain people do not wear shoes they could become infected with hookworm and, thus, these individuals could necessitate a cost of care. Moreover, the fact that certain people did not wear shoes is also a relevant economic risk factor; it will cost the provider today (if the patient did not wear shoes) and it will continue to cost the provider tomorrow (if the patient continued to not wear shoes).

This is a relatively easy situation. However, investigations into what is driving the economics of a given company's health costs may be much more difficult. Consider another example that is illustrative. An investigation into a given company may reveal unusually high expenses from ear infections in the dependents of company employees. What is driving this high incidence and the associated cost of care? A detailed analysis may reveal that more than half of the dependent children live in homes where their parents smoke. Second-hand smoke is a factor that predisposes children to ear infections. This identified character observation, parents smoking, is a relevant economic risk factor because its presence is driving the need for medical services to treat ear infections today and will continue to be a cost driver in the future unless parental smoking is modified.

Such investigative approaches are in stark contrast to prevailing practices of wellness programs that simply focus on identifying the presence of clinical risk factors and then attempt to modify those factors. Their logic is as follows. Clinical risk factors predispose a person to disease. If a person gets a disease it will cost money. Eliminating clinical risk factors will reduce the possibility of disease and, therefore, save money. As is evident, it is essential to discover clinical risk factors or characteristics of the individual or population in question that have economic value. This process requires the merging of multiple data domains and sophisticated algorithms in order to give statistical economic relevance for any given observation.

Finally, Venn diagram C illustrates the identification of disease states or conditions and relevant economic risk factors. For purposes of brevity, use of the term ‘disease states’ is meant to include any suitable disease condition as well. This Venn diagram illustrates the fact that certain disease states may be included in the relevant economic risk factors and are termed ‘a cost disease factor,’ whereas other disease states are not included. Consider an example that demonstrates this distinction. A given company may be evaluated, where it is determined that the company has an overall healthcare expenditure per annum of $6 million. The mean expenditure per individual for this company is $5,000. Further assume that about 20% of the healthcare expenses for this company (about $1.2 million) are attributable to heart disease. The company (in this example) includes 1,000 employees. Further analysis reveals that the total number of employees that spent $1.2 million on heart disease totaled 50. Thus, a minimal number of patients are directly responsible for the $1.2 million.

Now the important analysis begins, which involves determining why so few individuals are creating such huge healthcare expenditures for the company. First, the patients suffering from heart disease may be risk-stratified into appropriate categories (e.g. low risk, medium risk, and high risk). Note that such an environment is fluid; it is dynamic and constantly evolving. Such changing health factors, as well as the natural progression of a given disease, can readily be appreciated by medical professionals. Through diligence and a complete investigation, it may be revealed that six of the 50 patients had heart attacks and a corresponding bypass surgery. Further, by means of a cost stratification analysis, it may be discovered that these six individuals collectively cost the company almost $400,000. An in-depth evaluation may also uncover that, for these patients, these medical issues have generally been resolved. The conditions that caused the huge expenditures have been alleviated through their surgeries. After consulting with their physicians, it may be confirmed that these patients are stable, their health conditions have been successfully addressed, and the need for ongoing invasive treatment is non-existent over the next twelve months. Moreover, prior costs associated with these patients are not likely to recur. Thus, even these six patients, who were a huge healthcare expenditure for the company (representing almost 10% of the total cost), would be placed in the low risk heart disease category for next year's expense.

However, through the same in-depth analysis, it may be revealed that another patient in the heart disease group (“Herman”) had a severe heart attack, has a history of multiple hospitalizations, and, further, that he suffers from congestive heart failure. Herman's condition is not something that can be easily treated by a single event such as a bypass surgery. Herman has a demand for ongoing treatment. Not only is Herman most likely to see his overall health decline, there is a significant risk that Herman's future healthcare expenses will increase because of his condition. Accordingly, Herman would be designated in the high risk heart disease category for future expenses.

Referring back to FIG. 1B, within the disease state (as presented here) is a separate component: ‘ongoing treatment requiring hospitalization.’ Ongoing treatment requiring hospitalization may be completely absent (e.g. for the six patients identified above) or incredibly prevalent in various scenarios (e.g. for Herman) within the category of heart disease. This component is a cost driver. It is a relevant economic risk factor. It is an expense that is ongoing; it costs the company money today and tomorrow. Hence, within a specific disease state (e.g. heart disease, diabetes, lung cancer, etc.) there are relevant economic risk factors, which serve as the basis for ranking the patients into low, medium, or high risk categories. It is the underlying relevant economic risk factors within the disease state that are critical for determining future healthcare expenses. Therefore, cost disease state factors are relevant economic risk factors within a disease state.

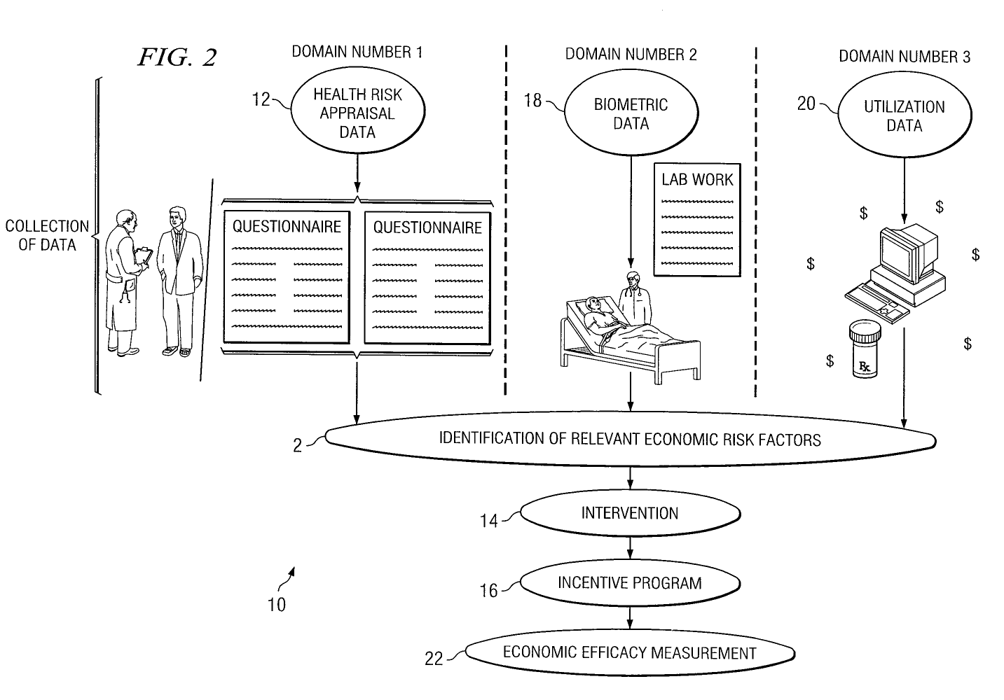

FIG. 2 is a simplified block diagram of a system 10 for managing healthcare expenditures in any given targeted environment. FIG. 2 offers some examples (offered for purposes of teaching only) that illustrate various activities and tasks that may be representative of each of the steps illustrated in FIG. 1A. Additional example activities and tasks are provided in subsequent FIGURES and offered throughout this document, but are clearly not exhaustive. Other alternatives, permutations, and substitutions are readily accommodated by the process and method of FIGS. 1A and 2 and are, therefore, certainly within the broad scope of the present invention.

FIG. 2 illustrates the collection of data retrieved from multiple domains in accordance with one embodiment of the present invention. System 10 may include three domains of information, which are used as a basis for the identification of relevant economic risk factors (i.e. the second step in the process). The domains include: health risk appraisal data 12, biometric data 18, and utilization data 20. System 10 may be provided for any suitable organization, company, corporation, institution, group, individual, or entity seeking to participate in the process and method of FIG. 1A.

System 10 allows for the collection of information in order to form three information domains. The information collected may be reviewed and processed in order to highlight relevant economic risk factors, which may then be used to develop a specific intervention 14 over a designated time period. Thus, the information collected in this first step may be used as a basis for subsequent steps to be completed in order to manage healthcare costs for the targeted population.

Once the data collection step is completed, the following steps may be completed: (step 2) identification of relevant economic risk factors; (step 3) design of interventions targeted to reduce the financial impact of these relevant economic risk factors; (step 4) an incentive program to promote participation of individuals in the interventions, which are targeted to modify these relevant economic risk factors; and (step 5) the measurement of the financial efficacy of the process in order to determine the value of the overall system.

Note that because the terminology associated with some of the elements of system 10 is malleable, it is helpful to offer some initial descriptions that address their meanings. As used herein, an intervention may be defined as an introduction of a variable (behavioral, chemical, process, etc.) that is designed to affect any cost expenditure for a target individual or group. Therefore, an intervention may include a change, addition, or modification to any relevant economic risk factor associated with the target. In the context of an intervention, a number of modules may be introduced to affect behaviors of the targeted individual or group. The term ‘module’ is defined below.

Within the structure of a given intervention, examples of a chemical modification (i.e. a module) for an individual may include changing a prescription from medicine A to medicine B or a change in treatment from Dr. A to Dr. B (or a treatment protocol being changed while remaining under the care of the same physician).

An example of an activity shift could include a recommendation to increase a level of physical fitness, to refrain from certain activities that pose an increased health risk, or to take precautions based on a particular set of symptoms or conditions identified for that particular individual. Other behavioral changes may stem from data or reports that suggest certain categorical groups (e.g. age, gender, race, etc.) or populations may be more susceptible to designated afflictions (e.g., a physician could recommend annual mammograms for women over the age of 35). In still other scenarios, the intervention could involve a process to be implemented, whereby the employee may be asked to interact with a nurse, immediately report cold symptoms to a primary physician, or log daily testing information in a journal. All of these modifications may be part of one or more designated modules for the target population. Such modules are discussed more fully below.

As used herein, health risk appraisal data 12 represents information that is extracted indirectly or directly from the employee or the treating physician. This information may be self-reported, for example, through a questionnaire or an interview (as illustrated in FIG. 2) that is completed by the target employee. Examples of such information include data relating to family history, current symptoms, previous surgeries, nutrition, smoking and alcohol habits, occupation, gene sequence, medication (past or present), or allergies. Note that because such information may reflect a specific trait of an individual or a population of employees, their specific constraints or conditions may be accounted for and accommodated.

For example, the fact that an employee is an investment banker in Manhattan, N.Y. may reflect a high stress level. Health risk appraisal data 12 could reveal such information, whereby the interview and/or the questionnaire could directly solicit this important fact. Thus, the interview and/or the questionnaire may be customized to address a particular population. Consider another example where the employee base is predominantly women. Appropriate questions for the interview and/or the questionnaire may then be associated with family history and breast cancer (note that gene sequence identification may be part of such an inquisition, as certain identified gene sequences do reveal a greater likelihood of breast cancer) or capabilities related to procreation potential. Numerous other examples of health risk appraisal data 12 are provided herein in this document for purposes of example and illustration. Alternatively, health risk appraisal data 12 could include any other suitable self-reported information, condition, symptom, or any other relevant fact, parameter, or piece of data that is relevant to the health of the individual or the group being evaluated.

As used herein, the term “module” includes any task to be completed by the targeted participants. The modules are designed after identifying the relevant economic risk factors associated with the target population. Hence, the identified relevant risk factors are used as the basis for configuring the modules, which are interactive and which specifically address the (potentially modifiable) targeted clinical risk factors, character observations, or disease states of the target population. Considerable time and effort may be expended in designing the precise modules that will yield the most beneficial results for the target group and, thereby, alleviate the healthcare costs for a given company. Thus, the modules are designed to reduce the healthcare expenses for a given individual or group, as determined by the identification of relevant economic risk factors. The modules may also achieve a reduction in healthcare expenses by modifying the choices of the individual so that the individual chooses new behaviors or abandons old behaviors that are costly.

Therefore, a module could include virtually any action, exercise, or assignment that may affect an individual's beliefs, feelings, thoughts, or behaviors. This is inclusive of an individual or a patient refraining from doing some action or intentionally not participating in certain endeavors. There could be a series of successive modules to be completed by an individual in a particular order, or the modules could be completed in a random fashion. A module is tailored specifically for a participant or a group of individuals and, therefore, modules are considerably flexible and malleable. A module may be completed during normal business hours (potentially under the supervision of an administrator), during non-business hours where the ‘honor system’ is employed, or anytime using a computerized system such as one described in more detail below.

Note that the modules are more process-oriented, as opposed to information-oriented, so that their focus is on the facilitation of change in the individual. The modules are designed to allow the user to acquire skills and life applications of the learned information. The user may be asked to respond affirmatively in order to address certain subject matter. In addition, the patient may be required to perform specific tasks. Rewards may then be given based on the performance of the modules by the individual, as he completes, applies, acquires, or participates in proscribed assignments within the modules.

A module could include educational tools, such as a booklet or computer program designed to address the illness, behavior, or issue presented by the target individual or group. For example, if the issue were stress management, a booklet could include information about proper diet (e.g. inclusive of caffeine restrictions), breathing exercises, time management, and sleeping suggestions. The booklet could include fill-in the blank questions that quiz the individual on the lessons learned.

The module could also solicit personal reflections from the individual. Note that such introspection is a powerful tool for addressing the patient's psyche at a fundamental level. Completion of question and answer sections could be part of the module, but probing deeper by asking difficult and private questions may prove far more beneficial. This is critical. Knowledge, by itself, does not necessarily change behavior. The individual needs to make a conscious decision to accept the knowledge and then incorporate these teachings into their own life. Asking thoughtful questions that query a person as to how they are feeling, thinking, and processing the presented information helps to foster their development.

Consider the following two questions that are illustrative of this concept. These questions could be provided in any potential module. Question 1: How do you feel about your current health self-assessment? What surprises you and what concerns you? Please explain. Question 2: Based on all of the information that you have learned so far in this module, what is your number one reason for wanting to take responsibility for your health? Such questions are far removed from simple fill-in-the blank questions or insignificant true/false questions.

A wise philosopher once noted: to know, and to not do, is to not know. Such an aphorism is relevant in the realm of healthcare. Slipping a pamphlet under the door of every employee of a company who has diabetes may not yield a change in behavior in these individuals. Facilitating change in the individual is paramount. For example, in the case of a diabetic individual, the critical issue is to not only get the individual to understand the value of blood sugar levels to their own wellness, but to make decisions that ensure that those blood sugar levels remain in an optimal range. Note that this recognition and application by the individual exhibits the knowledge and application components of the process being merged. After suffering an unfortunate incident or trauma (e.g. a seizure or a neuropathy), many diabetics might recount that they were made aware of a certain risk or a potential danger. For example, an individual who previously participated in a wellness program may explain, “Yes, I was once told of the dangers of failing to maintain my blood sugar levels. I remember completing a crossword puzzle about it.” Such a response elucidates the futility of many wellness programs. Healthcare expenditures have little to do with what people know or do not know. Instead, healthcare expenditures have far more to do with how people think, feel, believe, and behave, and, further, the choices that they ultimately make to live their lives. Thus, many of the modules presented herein are designed to facilitate the process of change so that an individual makes new choices in life that reduce the risk for disease or the cost of care of an existing one. Changing the thought processes, belief, and choices of the target individuals is key.

Modules can also be related to physical exercises to be completed by the participants of the target group. An honor system may be employed for such a module or the participant may wear some type of activity monitor (e.g. a pedometer for tracking walking, a heart rate monitor for tracking other activities, etc.). In addition, a module may include work completed using a computer and, potentially, monitored by an on-line administrator. A module could also simply be the completion or achievement of a specific goal. In the case of a person with high cholesterol, a reduction of the individual's cholesterol level by fifty points may signify performance or completion of the module. Other modules could include the ingestion of medication in the presence of a nurse or an administrator of the intervention. For example, a diabetic may be reluctant to take his proper insulin dosages and, therefore, present a significant financial risk for a company. A module could be designed specifically to address this problem, whereby a full month of consistent dosages (reflected by a nurse's log or by periodic measurement of blood sugar levels for this individual) reflects the completion of a module. The subsequent module for this individual could include a three-month period of consistent medication, which can be reflected by three months of consistent blood sugar levels being recorded in a table or chart verified by an attending nurse.

Other modules may be completed in a group setting. For example, if unplanned pregnancies are an issue for a company, a module could include female participation in a group meeting that includes women who previously experienced an unplanned pregnancy. Note that the group dynamic provides an opportunity for individuals to encourage each other in participating in the module. Thus, certain modules may solicit participation by an entire group of individuals for successful completion of the module. This group dynamic concept is a distinct issue that holds value; it is explained in greater detail below.

Other modules could implement the use of external sources. For example, one module associated with an unplanned pregnancy intervention could include regular attendance at Planned Parenthood meetings for three months, where information is regularly exchanged about contraception, proper nutrition, and exercise. Similarly, regular attendance at Alcoholics Anonymous could be required (for a specific period of time) for someone who is an alcoholic and who is also diabetic. Other variations and permutations in the design of the modules may be ascertained by simply focusing on the correctable and modifiable behaviors of the underlying target individual or group: behaviors which have economic significance.

As used herein, biometric data 18 reflects measured health information that is not necessarily self-reported. This information may be gathered from (or relate to) the employee and generally reflects physical data, which is measured. Biometric data 18 may relate to diagnostic information that could be provided in a laboratory report or gathered, for example, during the course of a magnetic resonance imaging (MRI) scan, in the context of evaluating a employee, or in performing some type of lab work (as illustrated in FIG. 2) or blood-work. In other scenarios, biometric data 18 may involve assessing body fat and blood cholesterol, lung capacity (e.g. using a flow meter), height, density and weight measurements, or any other suitable test or evaluation that yields some tangible result for an examining entity. In still other embodiments, this could include testing (e.g. psychiatric evaluations) that involves questionnaires, inkblot tests, etc. Alternatively, biometric data 18 could include any other suitable physical measurement, dimension, relevant health fact, parameter, or piece of data that may be collected by a physician, nurse, or representative authorized to do so.

As used herein, utilization data 20 refers to economic data that reflects financial information tied to the person or group being evaluated. This could include how much money is spent on pharmaceutical supplies, or some particular event such as a doctor visit or a trip to an emergency room at a local hospital. This cost sentiment is reflected by the illustration of Domain Number 3 in FIG. 2. Utilization data 20 may be solicited from a third party carrier or a third party administrator or, alternatively, through any other suitable entity. This may be inclusive of records searching in an appropriate database or file system. Utilization data 20 may reflect an economic event in which medical service triggered any type of fee. Such data is tied into costs incurred by a participant or by an employer on behalf of the participant. Alternatively, utilization data 20 could include any other suitable information or piece of data that may affect expenses for the individual or group that is being evaluated.

In the context of an example that includes the use of these three information domains (health risk appraisal data 12, biometric data 18, and utilization data 20), the following scenario is illustrative. A person may complete an interview session in which he answers truthfully that he has asthma and a history of heart disease in his family (this represents health risk appraisal data 12). He may then be tested using a flow meter that indicates he has limited lung capacity (this represents biometric data 18). He may also have his blood evaluated, which in this example yields that he has terribly high cholesterol (this represents biometric data 18). Finally, searching through a database or querying the employee may yield that he purchases several inhalers per month, that he was rushed to the hospital last year for an asthma attack, and that he is currently taking prescription medication to reduce his cholesterol (this represents utilization data).

In a general sense, Step 1 (or the collection of data) and Step 2 (identifying the economic relevance of that data) reveals the potential financial risk of the individual. An intervention can be subsequently introduced in order to provide modules that address the risk factors driving the cost of care. This allows for a clear and definitive plan of attack for this individual. Once the modules have been successfully completed, the overall value of the process may be displayed: comparing expenses before the intervention and expenses after the intervention using a statistically validated method of evaluation. This translates into a tangible result to be compared and validated for any interested party (e.g. the employer). Such a protocol avoids speculative claims or prognostications that may or may not prove truthful. This process produces a true bottom line result that can reflect changes in making comparisons (for example) year over year. This also allows for an easy identification of a change in value spawned by the process.

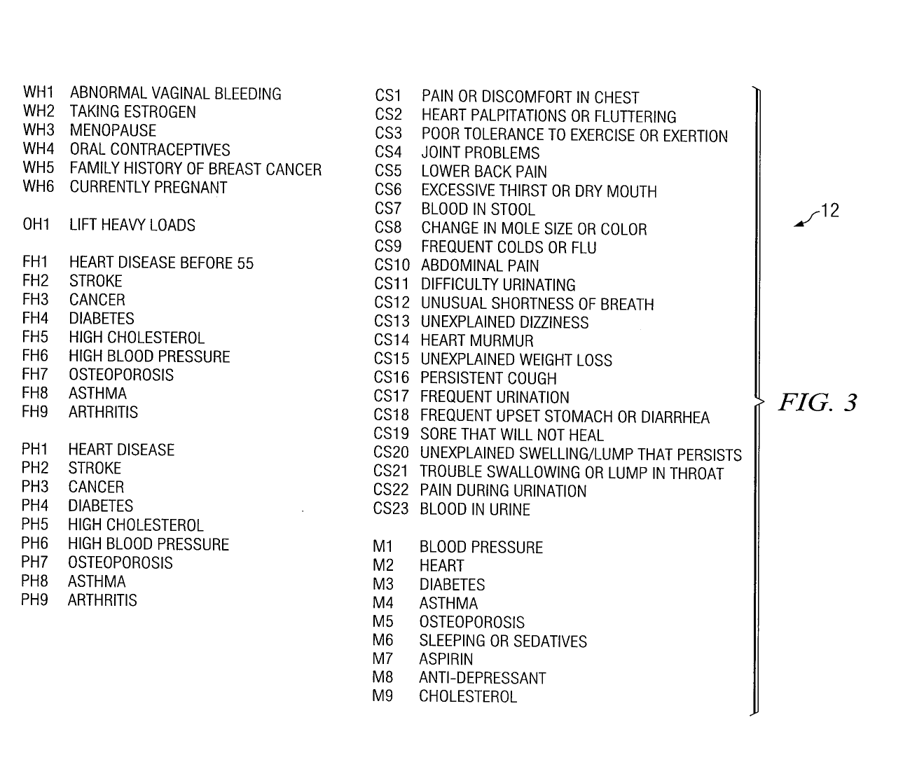

FIG. 3 is an example listing of health risk appraisal data 12. It is critical to note that such a listing has been offered for purposes of example and teaching only, and in no way should be considered exhaustive. Other health attributes can be readily accommodated by system 10 in accordance with particular needs or concerns. A series of codes are listed to the left of each of the data.

In operation of an example embodiment, health risk appraisal data 12, and biometric data 18 is collected for a targeted group of employees. Utilization data 20, which reflects actual dollars spent for a given time period for all employees and dependents, is subsequently acquired. The following list reflects what may be used to produce one example of economic efficacy: 1) employee and dependent identification number, 2) class code (employee, spouse, child), 3) amount billed, 4) amount paid, 5) ICD-9 codes associated with each employee and dependent, 6) CPT-4 codes associated with each employee and dependent, 7) date of initial contact with physician within a specific calendar year, 8) age, and 9) gender. Other information is acquired as needed by a specific provider or customer.

Referring back now to FIG. 2, once the data is collected from the three domains, relevant economic risk factors are then identified. This represents the second step in the process and method for managing healthcare expenditures. The purpose of the risk identification step is to discover relevant economic risk factors that reflect predictable events or conditions and, further, whose modification can lead to a reduction in healthcare expenses. This step is what Andrew Carnegie eluded to when he said: “find the cost.” The identification of relevant economic risk factors is the discovery process associated with finding the cost that drives expenses today and that will subsequently drive costs tomorrow. It represents a true economic driver. Modifying or eliminating that economic driver will directly affect a future cost. Relevant economic risk factors may be classified into three categories: 1) cost clinical risk factors; 2) cost character observations; and 3) cost disease states (as illustrated in FIG. 1B).

Let us explore what constitutes cost clinical risk factors. Consider the fact that medical research has determined that the probability of developing a disease is associated with specific risk factors. For example, there are generally five primary risk factors for heart disease: 1) smoking, 2) sedentary lifestyle, 3) obesity, 4) family history of heart disease, and 5) elevated blood lipids.

They are termed ‘clinical risk factors’ and are used to identify individuals who are at risk for developing coronary heart disease and a possible heart attack. Logically, modifications to these risk factors reduce the risk for disease development, as well as death and disability from a heart attack. What clinical risk factors fail to indicate about a person (who has those risk factors) is timeliness, when the heart attack will manifest itself, and the potential cost for the party that has the economic risk.

Thus, it may be accepted that a person will get sick because of risk factors that are present, but when a person gets sick and the cost attributed to the care of that illness is an entirely different question. It is because clinical risk factors fail to convey any sense of timeliness that clinical risk factors, in and of themselves, do not express economic value. A company may have 10% of its work force overweight, but what is the economic relevance of that finding for a company's healthcare expenditures next year? Not all clinical risk factors have economic value, nor are they equal in economic value. The challenge is to discover the economic relevance of a clinical risk factor.

Ideally, it is desirable to know not only if a person is at risk for disease, but when that person will get sick and what will it cost. If illness is inevitable, the associated cost is high, and it is likely to happen next year, then, the cost benefit ratio of a selected intervention makes early intervening protocols economically feasible for managing the healthcare costs of a population. A clinical risk factor becomes a relevant economic risk factor when it has been statistically documented to explain the current year's expense. In addition, it is a quantifiably predictive factor for next year's expenses. A clinical factor, so identified, becomes a relevant economic risk factor because it influences cost today and tomorrow. It may be referred to herein as a “cost clinical risk factor.”

There are generally two other kinds of relevant economic risk factors that may be identified. The first of these are cost characters. Recall that a character observation is a non-clinical finding or observation of the individual or group. If that finding is determined to have economic value, it is called a cost character. Once again, an economic driver has surfaced that has a statistical significance to drive cost over time. However, it is not necessarily a clinical risk factor like smoking. It is, in fact, non-clinical in nature. For example, suppose in the analysis of dollars spent for healthcare services it is found that 10% of the money is spent for emergency room visits. For a large company, this could mean millions of dollars. The question is, will the company spend 10% next year on emergency room visits? If so, what is driving these visits? Is there a cost character present, reflective in this disproportionate expense in the context of emergency room visits?

Further analysis may reveal that the nature of the visits (representing a whopping 50% of the emergency room visit expenses) are non-critical, do not result in hospitalization, and are associated with children. It may also be noted that most of the children involved in these emergency room visits have mothers who are working during normal working hours (e.g. 9:00 AM to 5:00 PM). After conversing with the mothers, it may be determined that such persons are only able to seek medical attention for their children after normal working hours and, thus, these women are not able to make simple appointments to see treating physicians. Unfortunately, with no alternative present, the women use the emergency room to seek ordinary and routine treatment for their children.

The cost character here is the lack of access to physicians and doctors during normal business hours. An emergency room visit increases the cost by a factor of 5 (five times). Hence, a $50 office visit is now transformed into a $250 emergency room expense. This lack of access to physicians will be present in the future (next year and subsequently) unless accessibility is modified. Accessibility is a cost character: driving cost today and in the future. In addition, it has been identified to have economic relevance. All cost characters, by definition, are relevant economic risk factors. Once a cost character has been identified, further analysis can define the interventions (inclusive of a number of modules) necessary to reduce the economic risk.

The last category of relevant economic risk factors that may be identified is the disease state or condition. In order to identify relevant economic risk factors within a disease state or condition, certain questions need to be asked. It is one thing to know that someone has diabetes; it is understood that he will have diabetes next year and the following year and so on. His disease process is generally predictive of cost, in and of itself. But not all diabetics cost the same to care for; some are more expensive than others. What then are the drivers of cost that could be attributable to human behavior and that are independent of the disease process? In other words, the disease is the disease. But how the individual relates to the disease (his behavior), is a completely different matter.

Suppose there are two diabetic individuals present at a given company. Both individuals are at risk for amputation. Each person has a peripheral neuropathy; the sensory nerves in their legs do not work properly. For example, the toes and feet could feel numb for both of these individuals. Each of these diabetics, in this example, also requires routine insulin injections.

Summer comes. One diabetic wears shoes. The other goes barefoot. Amputations are common amongst diabetics. Amputations generally take place as a result of an infection, which is usually secondary to a cut or an abrasion. The barefoot diabetic steps on a nail. As it turns out, in this example, he is not diligent in taking his insulin injection so his blood sugar runs at approximately 180 mg as compared to a normal blood sugar level of 100 mg. It can be readily appreciated that high blood sugar levels predispose a diabetic to bacterial growth.

Thus, in the context of this example, the two identified behaviors of one individual have predisposed this individual to any number of complications. His two behaviors—not wearing shoes and not routinely taking medication (resulting in high blood sugar levels)—have set him up for infection and a resulting amputation at a cost of $80,000.

In this example, the patient's non-compliance to take medication and lack of responsibility to take care of his feet (i.e. his own behaviors) become a cost character within his own disease process. His specific behaviors become relevant economic risk factors. These relevant economic risk factors are independent of the disease process. Relevant economic risk factors that are identified and that drive cost independent of the disease process are termed herein as “cost disease state factors.” These variables within disease states have been statistically determined to drive current cost and future cost and, if modified, reduce future healthcare expenses. As noted in the above example, modifying the relevant economic risk factors of the patient's two behaviors so that he wears shoes and becomes more compliant with his medication usage would significantly reduce the risk for a potential amputation and its associated cost.

Referring back to FIG. 2, consider the following example that illustrates one embodiment of the risk identification step. Company Alpha is desperate to curb its excessive healthcare spending. Company Alpha then authorizes implementation of a process that attempts to lower its healthcare spending. The first step of this process involves collecting information about the employees of Company Alpha. In this example, Company Alpha has an employee population of 5000, and spent $40 million on employee healthcare in the previous year. The driving factor behind the $40 million expenditure needs to be ascertained. Thus, the recurring relevant economic risk factors need to be identified within the employee population. For example, a simple audit could indicate that Company Alpha is spending significant money on high-risk pregnancies—accounting for approximately 25% ($10 million) of the total healthcare budget. Additionally, 20% of last year's healthcare costs (i.e. $8 million) might be related to cardiac-related problems. In addition, $4 million (i.e. 10%) may have been spent on emergency room visits.

After identifying these preliminary parameters, further inspection may then be needed to identify some of the underlying clinical risk factors, character observations, or disease states attributable to these expenditures. For example, are the cardiac-related expenditures a result of employees that continue to smoke, that live a sedentary lifestyle, or that have unacceptable dietary patterns? Moreover, what is the basis for the expenditures associated with the emergency room visits? The reasons underlying the unacceptably high utilization component need to be identified. Are these employees treating their local emergency rooms like after-hours clinics? Were all of the visits made in the last year truly necessary? Furthermore, why is Company Alpha spending so much on high-risk pregnancies? Are the pregnant mothers uninformed about issues such as smoking, drug addiction, and alcohol abuse? Are the mothers not getting adequate care during earlier stages of the pregnancy (i.e. in the first and second trimesters), as opposed to only being seen by a physician during delivery? Such important and probing questions focus on the specific root problem that has triggered the aforementioned excessive spending. Thus, these relevant economic risk factors are targeted and addressed in order to offer an optimal solution to the excessive healthcare spending of Company Alpha. Further, these relevant economic risk factors may be used in order to develop a specific intervention that fits the needs of the targeted population.

In a general sense, a highly intense investigation occurs within system 10 to find the costs and to reduce them. The role of detective is assumed in order to analyze patient data that is economically relevant. Consider another example case in Company Alpha where two employees, who are both diabetic, have divergent healthcare expenditures. The first patient (Paul) has healthcare costs associated with his recent neuropathy. The second patient (Peter) has recently suffered a heart attack. Paul's condition is generally associated with high blood sugar levels, whereas Peter's condition was influenced greatly by his smoking and high cholesterol. These two conditions were revealed in the first step of the process (i.e. the data collection step). Paul's condition caused only $2000 in healthcare expenditures for Company Alpha; Peter's heart condition necessitating bypass surgery may have cost $60,000 for Company Alpha. A further investigation may yield that Peter's cholesterol has dropped 100 points since his last doctor's visit (as a result of medication and regular exercise), but Paul's glucose levels are still being left elevated and unchecked. One person's healthcare costs (Paul's) might be about to skyrocket because of a potential amputation, whereas another's extraordinary healthcare costs of the previous year are most likely to disappear. If some arbitrary wellness or disease management company were only to look at last year's expenditures for these individuals, or to only review their age/demographical information, the anticipated or predicted cost of care associated with Peter and Paul and the necessary counter-interventions to abort these costs would go unnoticed. System 10 avoids such an inept analysis and, instead, places a greater emphasis on substantial investigative work in determining the true relevant economic risk factors. The investigation in identifying relevant economic risk factors may then be used as a basis to design interventions that curb the future costs associated with these present economic drivers. As Andrew Carnegie repeatedly quipped, “Find the cost and reduce it.”

Note that the present invention stands in stark contrast to current methodologies that address the problem of healthcare spending. System 10 offers a definite and unique architecture for providing a solution to the dilemma of healthcare spending. System 10 may identify fifty, sixty, or seventy relevant economic risk factors that could be used to offer insight into how healthcare spending can be decreased. Furthermore, system 10 can filter out economic risk variables that are not predictive of future healthcare costs and that only represent misleading indicators, which skew any predictions about future healthcare costs. In alternative scenarios, system 10 may only identify one or a few economic risk variables that are relevant to achieving lower healthcare expenditures.

System 10 executes a statistical analysis that illuminates the healthcare expenditures for a given company. This analysis allows a company to determine the true cost drivers of its healthcare expenses. The statistical analysis may process factors such as family, history, age, behavior, current symptoms, etc., some of these factors being represented by health risk appraisal data 12, biometric data 18, and utilization data 20. Following the initial step of data collection, the identity of relevant economic risk factors can be discovered. Appropriate interventions may be designed to reduce the financial impact of these relevant economic risk factors, and the future healthcare expenses for the company may be reduced accordingly.

Again it should be recognized that such a strategy in healthcare expense management is quite different from existing applications and approaches. Current healthcare cost management strategies focus primarily on financial solutions. A few look at clinical risk factors or disease states, but fail to identify relevant economic risk factors. For example, a wellness company may identify clinical risk factors such as obesity and sedentary lifestyle in a population and assume that the current healthcare expenditure may be explained by the presence of these factors. This same wellness company may also assume that healthcare costs will continue on their current path for company Alpha based on the fact that the employee population is fat and inactive. The analysis and conclusion both terminate with the identification of the obesity and sedentary lifestyle for the target population. Weak attempts to address this problem could include exercise posters and a lecture being given to all of the employees of company Alpha.

Such an approach is not only shortsighted, but it also offers no hope for Company Alpha to reduce their problematic healthcare costs. Furthermore, identifying a certain clinical factor (e.g., that certain individuals are obese and lead a sedentary lifestyle) does not yield the economic value (or relevance) of these factors. Consider a specific case in Company Alpha, where a twenty-two year old employee (Bill) leads a sedentary lifestyle, smokes, and last year cost Company Alpha $000 in healthcare expenses. Now consider Jerry, a sixty-four year old man who smokes, is sedentary, was hospitalized last year for a respiratory condition, takes breathing medicine, and thinks people just get sick. Last year Company Alpha spent $10,000 on Jerry.

In this example, the additional factors of age, hospitalization, medication, personal belief system, and history of high cost gave economic relevance to the clinical risk factors of smoking and obesity. Jerry is at risk to cost company Alpha significant dollars next year while Bill is not. As a result, bringing multiple interventions to Jerry becomes a primary objective while fewer resources could be spent on Bill.

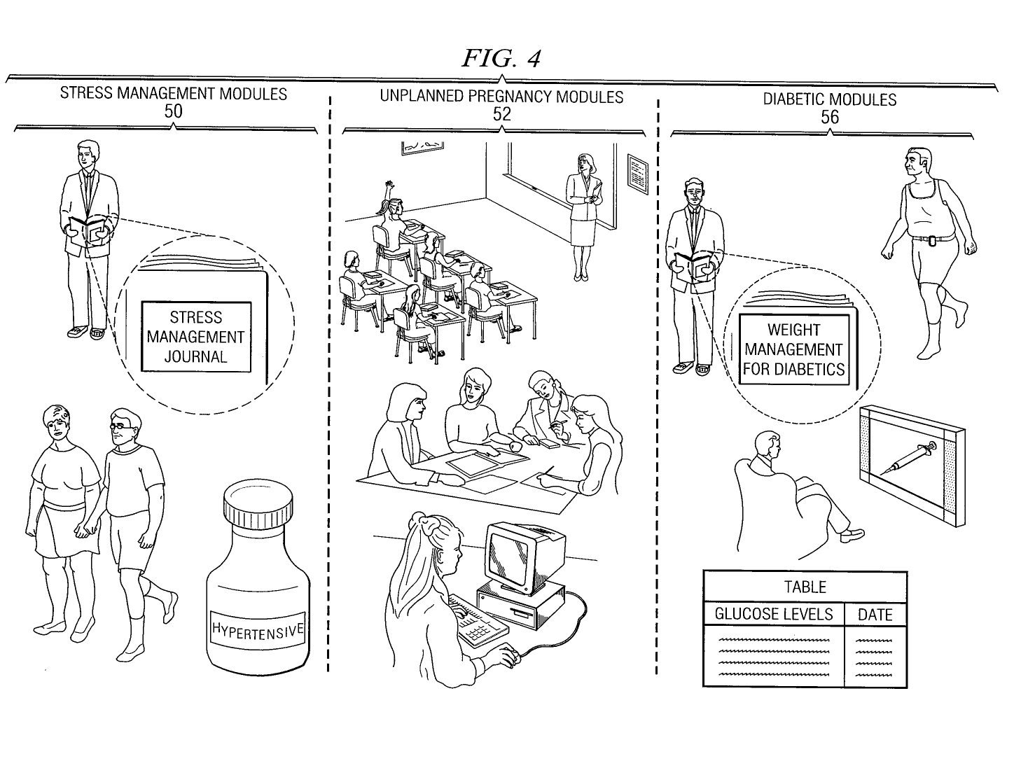

FIG. 4 is a simplified schematic diagram that illustrates a number of modules that address specific problems identified as relevant economic health risk factors. Recall that once a relevant economic risk factor has been identified, a specific intervention may be introduced that is designed to modify the risk factor and create an economic yield. The intervention can be directed toward any risk category: cost clinical risk factors, cost disease state factors, or cost characters. For example, if high blood pressure or high blood sugar is discovered to be a cost clinical risk factor in an employee population, an intervention would be applied (e.g. weight management) to that population to reduce the economic impact of obesity. Similarly, a cost character intervention could address factors such as generic drug purchases or treatment compliance.

The proposed interventions are generally of two kinds: behavioral based and non-behavioral based. Consider the case where there are high costs associated with recurrent emergency room visits for employees of Company Alpha. A non-behavioral based intervention could be designed to offer a 24 hour ‘doctor-on-call’ line so people could call a physician if they were sick and thought they might need to go to the emergency room. A behavioral based intervention or module could add an interactive journal designed to facilitate a change in how to behave toward the use of emergency medical services, skills on how to evaluate acute medical events, etc. Combining one intervention to change behavior with another intervention to change a point of service or a level of care optimizes economic efficiencies.

FIG. 4 illustrates one series of example modules that include a set of stress management modules 50, a set of unplanned pregnancy modules 52, and a set of diabetic modules 56. The specific modules may include any exercise or task to be completed by the targeted participants. The modules are designed after identifying the relevant economic risk factors associated with the target population. Hence, the identified relevant economic risk factors relate directly to the design of these example modules of FIG. 4. The modules address modifiable economic risk factors associated with the target population that lead to excessive healthcare cost.

The first set of modules address stress management. The ‘STRESS MANAGEMENT JOURNAL’ booklet illustrated in FIG. 4 could include information about proper diet (inclusive of caffeine restrictions), breathing exercises, and time management suggestions. The booklet could include fill-in the blank questions that quiz the individual on the lessons learned. The booklet could also solicit personal reflections from the individual. Completion of question and answer sections could be part of the module booklet, but more substantive feedback could be required from the individual. Such feedback may prove more beneficial as the feedback delves into significant behaviors that affect that individual's actions and, thereby, his healthcare costs.

Stress management modules 50 also include physical exercises to be completed by the participants of the target group. This is illustrated in FIG. 4 by the couple completing a walk. An honor system may be employed for such a module, or the participant may wear some type of activity monitor (e.g. a pedometer for tracking walking, a heart rate monitor, etc.). In most cases, the exercise that is proscribed should be completed consistently over a period of time (e.g. a month, three months, etc.). Other modules could include the ingestion of medication in the presence of a nurse or an administrator of the intervention. In the context of stress management and hypertension, an antihypertensive regimen (e.g. Catapres, Wytensin, Apresoline, Hytrin, etc.) is also assigned for this individual through a corresponding module, as illustrated in FIG. 4.

Unplanned pregnancy modules 52 may include various modules, which are similarly designed to affect healthcare costs associated with this group of individuals. In this example, these modules include work that may be completed with a computer and, potentially, monitored by an on-line administrator. The computer module provides an educational tool to be used by the participants in order to better understand pregnancy risks and contraception. Note that such a module could include a significant amount of reflective writing. Simple knowledge of being aware of the present risks for an unplanned pregnancy is not enough. The intent of this module is to help the individual actually process the information that is being presented and, further, to facilitate behavior (based on the knowledge learned) that will translate into a cost savings in healthcare expenditures.

Other modules may be completed in a group setting, as illustrated in FIG. 4. In the context of this example set of unplanned pregnancy modules 52, individuals may participate in a group meeting that includes mothers who previously experienced an unplanned pregnancy. Other modules could implement external sources. For example, one module associated with an unplanned pregnancy intervention could include regular attendance at Planned Parenthood meetings for three months, where information is regularly exchanged about contraception, nutrition, exercise, finance, etc. in the context of unplanned pregnancies.

Diabetic modules 56 could include a number of modules that are specifically designed to address health risk factors associated with this unique group. In this example, a booklet entitled ‘WEIGHT MANAGEMENT FOR DIABETICS’ is used to facilitate the changes in personal behavior necessary to achieve weight loss. Other booklets for diabetics could outline the importance of exercise. For this group of participants (or for a given individual in the group), walking exercises are to be completed. The individual illustrated in FIG. 4 has a pedometer on his waist that tracks the number of steps he takes. This information can then be verified by an administrator or simply downloaded into a computer or a database.

Modules for this individual, in this example, also include a documentary about diabetes to be watched by the individual. The movie could be accompanied by a follow-up exercise that solicits feedback from the individual. This could take the form of a simple interview or an actual test. A module could also simply be the completion or achievement of a specific goal. In the case of a diabetic person with high cholesterol, a reduction of the individual's cholesterol level by fifty points may signify the successful completion of an assigned module. In the case of a diabetic, a table (shown in FIG. 4) is to be used to monitor glucose levels. For example, a diabetic may be reluctant to take his medication. Therefore a module could be designed specifically to address this problem, whereby a full month of consistent dosages reflects the successful completion of a module. Thus, successful performance of this module may include consistent glucose levels being achieved by the individual and properly recorded in the table.

It is imperative to note that the modules of FIG. 4 only offer one simple example of how an intervention may be introduced to the target group. The specific modules of FIG. 4 may readily be replaced with any other suitable module that targets specific targeted clinical risk factors, character observations, or disease conditions of the individual, which were determined to be economically relevant in the preceding step of the process. Moreover, modules could be completed in a specific manner (inclusive of timelines and deadlines) such that the expected result is achieved. Considerable flexibility is provided by these modules as they are tailored to meet the exact needs of the individuals in the target group. It can be appreciated that the module arrangements presented here are arbitrary, as they have been only used for purposes of teaching. Accordingly, any module configurations offered herein in this document should be construed as such—simply one example of the millions of possible combinations and arrangements that may be used.

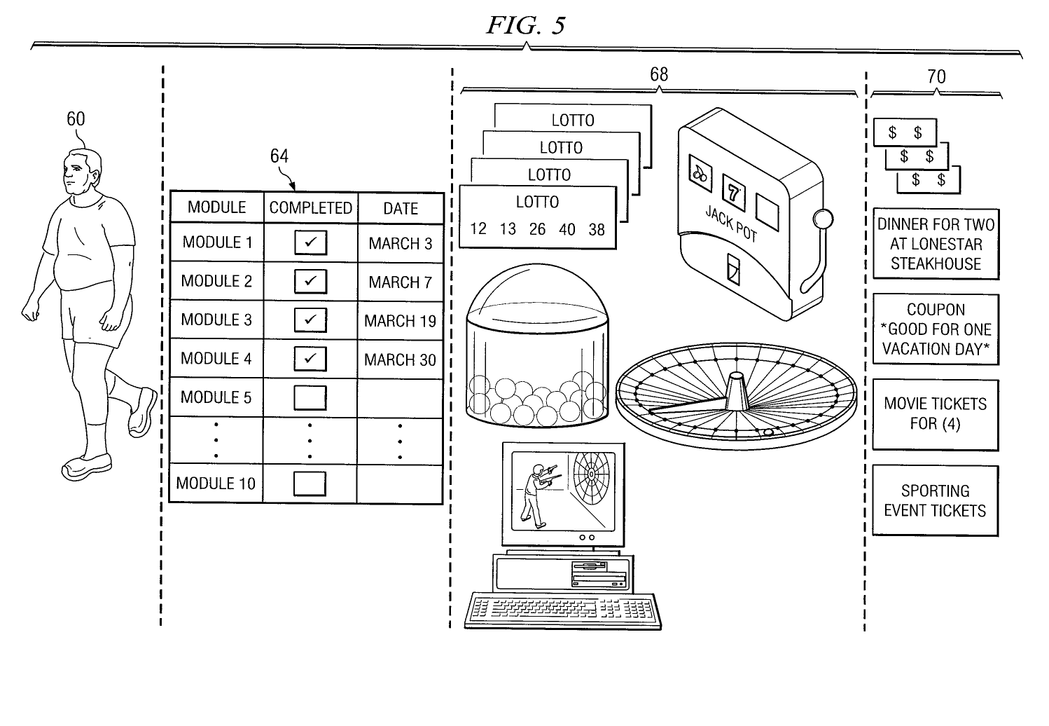

FIG. 5 is a simplified schematic diagram illustrating the interaction between step three (i.e. the intervention) and step four (i.e. the incentive program). Note that higher economic yields are obtained if people have an incentive to engage in a desired behavior. The basic components of the proposed incentive program could include: (1) a merit reward system that is linked to behavioral modules; (2) combining the merit system with a gaming system; and (3) translating the merit and gaming system into an economic reward system.

Note that many employees may be reluctant (for whatever reason) to participate in any level of the proposed wellness process. Consider the example where a company is somewhat segmented because of recent mergers or because of the division between union and non-union employees. An effective incentive program may be put in place to address this problem in order to encourage participation. In general, an economic reward is offered to solicit involvement in the program.

Consider one example where the behavior to be addressed is stress management. Employees may receive a behavior module that is designed to alter the way in which employees manage their stress. Then they are rewarded for the completion of each module and continue to apply these practices in their life and to acquire skills for managing their stress. Each employee can earn merit points (or tickets, coupons, vouchers, etc.) depending on his diligence and efforts.

Merit points allow the employee to earn opportunities or chances that are required to participate in the gaming system. For example, if fifty merit points were earned (e.g. through completion of several modules), this could allow the individual to have the chance to play (or pull) the gaming slot machine five times. Additional points could then be won (called reward points) during play of the slot machines. The five pulls could win twenty-five more points. So the point total is now seventy-five points. Merit points are generally not lost; they are used for the opportunity to win additional points. The individual might win no additional points or significantly more points during the gaming opportunity. The more merit points earned (through individual efforts), the more chances given to play the games and win additional reward points. Note that there could be a series of games (e.g. game #1, game #2, game #3, etc.) before a final payoff occurs. This could provide an ability to translate the total number of earned points into currency to buy merchandise. The total number of earned points could also culminate with a lottery system as described herein. The reward points could be given the same value as the merit points or provided as only a fraction of a merit point. Any suitable combination of reward points and merit points may be made in order to correlate these points to some type of reward (e.g. cash, prizes, etc.).

In the final step of this example, the total number of points accumulated during a given time period (e.g. one month), is then translated into lottery tickets. If 200 total points have been accumulated and 50 points buys one lottery ticket, then the individual could then pick 4 lottery tickets. Tickets may then be placed into a common pool where a drawing occurs. Multiple winners could then be randomly selected and rewarded with cash or prizes. The design of the system is to produce a statistically significant number of winners that entices the greatest number of employees to participate in the applied behavioral modules.

The central theme associated with any such scenario is that a relationship exists between the designed modules and the merit point system, which fosters completion of the modules. Viewed from a different perspective, the incentive program comprises merit, opportunity, and reward. Hence, the modules could include other behavior patterns or behavioral goals. Thus, a module could be designed to curb absenteeism, whereby merit points are earned by the individual by achieving a certain level of attendance at work. The employee could then be rewarded (based on accumulated merit points) with slot machine pulls, a lottery system opportunity, etc. as outlined herein.

FIG. 5 offers an example scenario where the issue of sedentary lifestyle is being addressed by a number of modules. An individual 60 is represented and has been assigned a number of modules 64 to be completed in a given term. Merit points can be earned for completion of the assigned modules. Merit points may then be correlated to gaming opportunities. A set of potential gaming opportunities 68 and potential rewards 70 are also illustrated in FIG. 5. In this example, twenty merit points could be earned for twenty thousand steps (through walking exercises) completed by individual 60. In addition, completion of a workbook could earn fifty points for individual 60. FIG. 5 illustrates that these two modules were completed on March 3rd and March 7th respectively. Additionally, in this example scenario, individual 60 completed computer modules #3 and #4 on March 19th and March 30th respectively. March 30th represents the end of the term for Company Alpha. Thus, a gaming opportunity could be provided on April 1st (or soon thereafter) for all those individuals who earned merit points during this one-month time interval.

Gaming opportunities 68 could include: a lottery, a roulette wheel, a slot machine, a computer game, or a bin of balls that represent prizes (or additional points) to be won. Other employees may be required to observe the gaming opportunities to encourage future participation. Other potential gaming opportunities could include raffling contests, card games, BINGO, or any other game of opportunity, chance, or amusement. These gaming opportunities (provided in the context of step four) correlate to participation in the assigned modules (step three), which were developed specifically as a result of the identification of relevant economic risk factors (step two of the process).

Gaming opportunities 68 can then yield one or more rewards illustrated in FIG. 5. For example, in the context of one scenario, gaming opportunities 68 could offer rewards such as: dinner for two, movie tickets for four, a coupon for one vacation day at work, sporting event tickets, or cash. As described throughout this document, other rewards and reward scenarios and arrangements could readily be accommodated by the present invention.

In an alternative embodiment of the incentive program (i.e. step four of the process and method), a bilateral incentive model may be used. This embodiment creates multi-level incentives to motivate individuals within a targeted group to participate in wellness program activities (e.g. modules, completion or submission of requested data, etc.). Underlying this process are principles of random rewards, opportunities to win valuable prizes, and prospects to participate in games of chance for those who qualify through their own personal performance.

In this example, which is offered for teaching purposes, the specified group may be identified both as a whole (e.g., an entire employee population of a company) and by specified component parts, or various divisions of the company (e.g., components and divisions may be selected by job description, such as sales, accounting, manufacturing, etc., by location or worksite, or by any other means of grouping the individuals). As used in this example, the term “population” represents the entire group, and the term “divisions” represents the designated component parts.

The modules may specify various behavioral modification tasks (as identified extensively above), such as completing a health questionnaire, performing specified tasks, or engaging in modules. Each task that is completed entitles the individual to a reward based on the merit of having engaged in or completed that specific task. This merit reward may be in any format that has no value of its own (other than entitling the individual to participate in the random reward opportunity phase of the program). Such merit rewards may be points, tickets, or any other method of indicating successful performance of the module or specified task.

In this alternative embodiment, all of the earned tickets for a specified task are entered into the random reward game. This may be any game of chance, such as those illustrated in FIG. 5. For example, the game may be a drawing, an Internet slot machine, a roulette wheel, or any other mechanism to randomly select a winner. A single game may also yield multiple winners of prizes in descending values. These random reward prizes may be in a variety of forms, such as cash, credit for purchasing products or services, travel, extra vacation days, etc.

One aspect of the bilateral approach to motivating participation in wellness activities is the appeal to personal passion to win a free prize without risking a wager in the process. Another aspect of the bilateral motivation approach is one of peer pressure or group dynamics. If during the course of time for completing a specified wellness task, the percentage of participants within one of the “divisions” reaches an established minimum threshold, then each individual who participated in that division may receive an extra ticket for the game of chance. In this manner, there is one incentive to personally earn a chance to play and to win, and a second incentive to encourage one's teammates within the group. By group participation in reaching the designated percentage, those individuals double their chances of winning through the efforts of the participants of all the other groups. Thus, this structure provides a bilateral approach to motivation.

A variation of this second aspect of the bilateral approach may be used as an alternative to the process described in the preceding section, or in combination therewith. If during the course of time for completing a specified wellness task, the percentage of the entire population reaches an established minimum threshold, then the amount of the grand-prize, and/or any of the lesser prizes for that game may be increased. Thus, the same peer pressure principle may be used to generate interest in participating by providing increased chances to win as well as increased winnings.

During the course of the method and process proposed herein, there may be multiple periods of time during which specified tasks may be completed to earn a ticket. Consequently, there may be a corresponding number of games for random rewards. As the population becomes aware of periodic winners, the influence of the bilateral incentive plan should increase from both the personal passion to win, as well as from the peer pressure to participate as a group.

In yet another alternative embodiment, a more direct approach to the relationship between the modules and the gaming opportunities may be achieved. For example, a module could be used to motivate any person to come to work (i.e. place of employment). Thus, a more simplistic module design could involve curbing absenteeism. Hence, behavior objectives could be used to encourage an employee's attendance at work. Such behavioral objectives are clearly within the scope of the term ‘module.’ In such a scenario, the underlying relevant economic risk factor being addressed is absenteeism. The module is provided to motivate the employee to be present at work on a regular basis. The completed modules would then readily translate into merit points that are used for the gaming opportunities or for a reward.

In still other alternative embodiments, the modules may be used to curb healthcare expenditures in yet another more direct fashion. Modules could be provided that motivate an employee to conserve healthcare spending over a given time period. Thus, the patient behavior again is being targeted through a reward for reducing risk that minimizes the consumption of economic resources. Therefore, achieving a reduction in medical expenditures over a given time period may yield merit points that can be used in a gaming opportunity. In one example, the gaming opportunities may be supplanted with the company paying the insurance costs normally incurred by the patient. This includes any implementation in which a third party performs this task. Thus, the company could pay the insurance premiums, deductibles, or any other expense that would otherwise be paid by the employee. This would offer a more straight-forward approach for any individual wishing to participate in some form of an intervention.

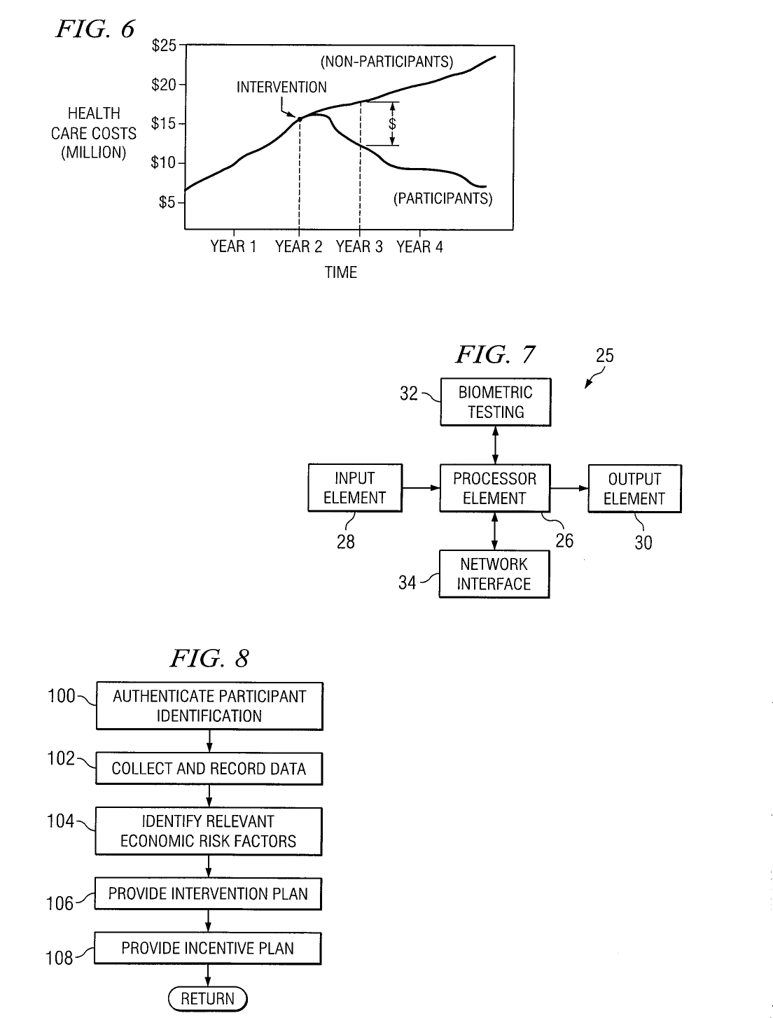

FIG. 6 is a simplified graph illustrating an example of how system 10 could be used to measure the economic efficacy in the context of an intervention that could include any number of modules. Before turning to FIG. 6, consider that the overall objective of any proposed healthcare solution is to improve the health status of the employee and to reduce utilization expenses of healthcare services. To be deemed valid, the success of the solution that is applied to a population must be measured.

Note that there is an ever-increasing need in healthcare to have the ability to measure not only the clinical benefit of treatments, interventions, and practices, but to measure the financial benefit as well. Does a disease management intervention, which uses as its core practice the process of sending brochures to employees with diabetes, making a 24-hour nurse hotline available, and sending a letter to doctors about what tests to run and what parameters to monitor truly reduce the cost of care? Such a question sparks controversy, and it may be debated forevermore. However, what is not contentious is that in having no ability to measure such an activity or strategy, it is impossible to know its efficacy. Thus, the healthcare industry currently fails to offer a metric that may be used to achieve such measurements. Despite this obvious failing, many companies continue to engage in the practice of disease management, and also continue to be exceptionally profitable. In many scenarios, these practices offer a baseless hope and a set of random solutions to those who are ailing and desperate for an effective treatment.

The present invention addresses these inadequacies and deficiencies by offering a true and valid measurement for the proposed interventions. Turning to FIG. 6, the graph of FIG. 6 is a function of healthcare costs for Company Alpha (provided on the y-axis), and of time (provided on the x-axis). At year two, an intervention is introduced in this scenario. The intervention addresses the employees of Company Alpha through a series of modules and can include any suitable changes in process, behavior, chemicals, etc., as identified throughout this document.

Year two signifies the introduction of the intervention, as well as the clear divergence in healthcare costs associated with participants versus non-participants. As evidenced by the graph, participants are achieving better wellness and health as a result of the interventions that have been instituted to affect cost expenditures. In contrast, non-participants are following the projected trend (based on previous years), as they are accounting for more healthcare costs for Company Alpha.

Note that, in addition to being able to clearly see the disparity in these two lines, what is possibly more important is the measurable increment in costs from year two to year three. This is provided on the graph and represented by a dollar sign. This incremental value decisively elucidates the actual savings in cost expenditures (over the course of one year) as a result of the intervention (and, thereby, the modules). The cost savings are augmented in subsequent years, which also may be readily measured in accordance with teachings of the invention. Thus, any interested party may be able to identify a tangible and genuine efficacy value associated with the intervention.

The proffered measurement allows the payer to see the financial benefit of the system and to identify an earnest return on investment. For example, consider a company that had 2000 employees and its healthcare expenditures were $12 million per year. Suppose 1500 employees participated in the program and the cost savings between those who participated and those that did not was $1200. This means that for each employee who participated in the process, the company saved $1200 or a total of $1.8 million. The determination of financial efficacy of any proposed process or method is a critical component because, without this, it would be impossible to demonstrate its value.

In a general sense, the graph of FIG. 6 (and the process of system 10) offers a yardstick to measure the economic efficacy of an intervention. Proven financial efficacy is particularly valuable in the field of health and medicine, where speculation and baseless predictions are common. The process provided by teachings of the invention statistically validates the economic efficacy of an intervention applied to an individual or individuals. This could also provide a return on investment ratio, again revealing the efficacy of any given intervention and accompanying modules.

FIG. 7 is a simplified block diagram of a data processing system for delivering and administering certain aspects of the invention. In one embodiment, the data processing system, referred to herein as a health station 25, comprises a processor element 26, an input element 28, an output element 30, biometric testing element 32, and a network interface 34. Health station 25 may represent a server, client, or peer data processing system, depending on context and applicable tasks. In certain embodiments, input element 28 and output element 30 may be combined into a single user interface element, such as a touch-screen display or kiosk. Moreover, health station 25 generally includes a means for authenticating a user (e.g., a participant in an intervention). The means for identifying a user may include a card reader, fingerprint scanner, or any other well-known software or hardware authentication system.

Health station 25 provides a means for delivering an intervention to a given population, and thereby modifying risk factors that are driving costs. Moreover, health station 25 may provide a means for administering an incentive program associated with the intervention. Health station 25 may authenticate a participant, track participation, store relevant data, report intervention progress or incentive program status. A data processing system such as health station 25 also may be configured with software, application specific integrated circuits (ASICs), or other means to implement an algorithm associated with steps identified in FIGS. 1A and 2.

In certain embodiments, network interface 34 may be coupled to a communications network (e.g., the Internet) or any other communicative platform operable to exchange data or information with other data processing systems. The provided communications network may alternatively be any local area network (LAN), metropolitan area network (MAN), wide area network (WAN), wireless local area network (WLAN), virtual private network (VPN), intranet, plain old telephone system (POTS), or any other appropriate architecture or system that facilitates communications in a network or telephonic environment.

When the communications platform is network-based, the functions of health station 25 may be distributed across several health stations or data processing systems. For example, participant history and biometric data may be collected through a first health station 25, and then transmitted to a second health station 25, server, or other data processing system at a remote location for storage or further processing. Moreover, several health stations may be located at various locations to service geographically distributed populations, and a network-based health station 25 provides a means for a participant to remotely input, change, or update health information, as well as participate in certain intervention activities.

To illustrate some of the advantages of health station 25, assume that relevant economic risk factors for coronary heart disease of a given population have been identified, and that an intervention has been designed to reduce these risk factors. More particularly, the relevant economic risk factors have been identified as obesity, high blood pressure, and a diet high in saturated fat, and the intervention includes providing a diet that is low in saturated fat and track participation, ensuring that all high blood pressure participants are on medication or losing weight and responding to treatment, and providing instruction for weight management and tracking results. Moreover, assume that an appropriate incentive program has been designed that requires each participant to measure weight once a month and measure blood pressure twice a month. In addition, each participant must view a series of educational videos on heart-healthy nutrition, and keep a dietary record. Finally, assume that each participant is given a weight management plan and must record progress weekly.

In this example scenario, health station 25 facilitates the delivery of the intervention and administration of the incentive plan. For example, health station 25 may require each participant to provide authenticating credentials, such as an identification card, fingerprint, or password. Moreover, health station 25 may provide a convenient touch-screen interface that allows a participant to activate the educational videos as streaming video, and may provide an interactive weight management plan. Health station 25 may further provide an interface that allows a participant to create and manage the dietary record, and record compliance with the weight management plan. Biometric testing elements 32 may measure and record the participant's weight and blood pressure. Additionally, health station 25 may be programmed or otherwise configured to query the participant for information indicative of compliance, such as whether or not the participant is taking medications as prescribed. Finally, the information collected may be transmitted to a remote health station 25 or other data processing system via network interface 34, where it may be stored, tracked, and analyzed. A participant may then review a progress report and the status of any rewards or incentives.

It should be noted that the internal structure of the system of FIG. 7 is malleable and can be readily changed, modified, rearranged, or reconfigured in order to achieve its intended operations or additional operations. Accordingly, processor element 26 may be equipped with any suitable component, device, ASIC, hardware, software, processor, algorithm, read only memory (ROM) element, random access memory (RAM) element, erasable programmable ROM (EPROM), electrically erasable programmable ROM (EEPROM), or any other suitable object that is operable to facilitate the operations of processor element 26. Considerable flexibility is provided by the structure of processor element 26.

FIG. 8 is a flow diagram that illustrates one embodiment of an algorithm associated with a health station, which implement various steps described above with reference to FIG. 1A. This algorithm is described from the perspective of a network-based health station, in which the health station is coupled remotely to a server, data processing system, or second health station through a network. In general, a health station requires each participant to be authenticated. While the algorithm contemplates use of a wide variety of authentication algorithms and systems well-known in the art, one such means includes an identification card having a magnetic stripe or other computer-readable medium. Each participant may be issued such an identification card, which uniquely identifies the participant to a health station. Thus, in step 100 the remote health station collects the participant's identification, authenticates the identification, and records the identification. In step 102, the health station collects and records health-related data from the participant. Here, the health station may interactively prompt the participant for the information, such as a family health history, or may prompt the participant to activate a biometric testing element to measure certain information. In step 104, the health station identifies one or more relevant economic risk factors from the health-related data, using any of the techniques, processes, or systems described above with reference to FIGS. 1-7. In step 106, the health station provides an intervention plan based on the relevant economic risk factors. Again, the health station may be configured to implement any of the techniques, processes, or systems described above to provide the intervention plan dynamically. Alternatively, an administrator may store several static intervention plan options in the health station, and the health station then selects an intervention plan from these options based on the risk factors. Step 106 may further comprise steps for delivering elements of the intervention (such as streaming video), tracking participation (e.g., requiring participant authentication before and after viewing a video), storing relevant data, and reporting intervention progress. In step 108, the health station provides an incentive plan to the participant. This step may further comprise tracking and reporting the participant's incentive status, and optionally, delivering certain incentives.

Note that the example embodiments described above can be replaced with a number of potential alternatives where appropriate. The processes and configurations discussed herein only offer some of the numerous potential applications of the invention. The elements and operations listed in FIGS. 1A-8 may be achieved with use of system 10 in any number of contexts and applications. Accordingly, communications capabilities, data processing features and elements, suitable infrastructure, adequate personnel and management, and any other appropriate software, hardware, or data storage objects may be included within system 10 to effectuate the tasks and operations of the elements and activities associated with correlating an economic relevance to health variables. In addition, FIG. 7 provides only one example of a suitable processing and communications platform for health station 25. In certain embodiments, all of the elements of FIG. 7 may be provided in a single fabricated electronic element or module.

Certain features of the invention have been described in detail with reference to particular embodiments in FIGS. 1A-8, but it should be understood that various other changes, substitutions, and alterations may be made hereto without departing from the sphere and scope of the present invention. For example, although the preceding FIGURES have referenced a number of economically relevant health risk factors, any suitable characteristics or relevant parameters may be readily substituted for such elements and, similarly, benefit from the teachings of the present invention. These may be identified on a case by case basis, whereby a certain patient may present a health risk factor while another (with the same condition) may not. Thus, a statistical relevance may be identified for one group, but not another who appears to be similar.

Although the present invention has been described with several embodiments, a myriad of changes, variations, alterations, transformations, and modifications may be suggested to one skilled in the art, and it is intended that the present invention encompass such changes, variations, alterations, transformations, and modifications as fall within the scope of the appended claims.

What is claimed is:

1. A method for managing health risks, the method comprising:
identifying one or more relevant economic risk factors from health-related data collected from a person;
providing an intervention plan to the person based on the relevant economic risk factors;
authenticating an identity collected from the person at a remote location;
exchanging data related to the intervention plan with the person at the remote location; and
providing an incentive to the person for complying with the intervention plan.

2. The method of claim 1, wherein the health-related data comprises health risk appraisal data.

3. The method of claim 1, wherein the health-related data comprises biometric data.

4. The method of claim 1, wherein the health-related data comprises utilization data.

5. The method of claim 1, further comprising tracking changes in the health-related data; wherein the incentive is based at least in part on changes that comply with the intervention plan.

6. The method of claim 1, wherein providing the intervention plan comprises providing one or more intervention activities that reduce the relevant economic risk factors.

7. The method of claim 6, further comprising tracking the person's participation in the intervention activities; wherein the incentive is based at least in part on participating in the intervention activities.

8. The method of claim 6, further comprising tracking the person's participation in the intervention activities by verifying the identity of the person before beginning the intervention activities and after completion of the intervention activities; wherein the incentive is based at least in part on participating in the intervention activities.

9. The method of claim 6, further comprising tracking the person's participation in the intervention activities by verifying the identity of the person before beginning the intervention activities and after completion of the intervention activities; wherein the incentive comprises a merit point system based at least in part on participating in the intervention activities.

10. A system for use in a health management program, the system comprising:
a user interface for receiving a user identity from a person, and collecting data related to an intervention plan for reducing one or more relevant economic risk factors identified from health-related data associated with the person;
a network interface for exchanging data related to the intervention plan with a remote system;
a tracking component for tracking compliance with the intervention plan; and
a processing component for calculating a credit to the person for changes that comply with the intervention plan.

11. The system of claim 10, wherein the health-related data comprises health risk appraisal data.

12. The system of claim 10, wherein health-related data comprises biometric data.

13. The system of claim 10, wherein the health-related data comprises utilization data.

14. The system of claim 10, wherein the user interface delivers to the person one or more intervention activities associated with the intervention plan.

15. The system of claim 14, wherein the credit is based at least in part on participating in the intervention activities.

16. The system of claim 14, wherein the tracking component further tracks the person's participation in the intervention activities by verifying the user identity before beginning the intervention activities and after completion of the intervention activities; and wherein the credit is based at least in part on participating in the intervention activities.

17. The system of claim 14, wherein the tracking component further tracks the person's participation in the intervention activities by verifying the user identity before beginning the intervention activities and after completion of the intervention activities; and wherein the credit is derived from a merit point system based at least in part on participating in the intervention activities.

18. A system for use in a health management program, the system comprising:
two or more health stations for determining identities of users participating in an intervention plan for reducing one or more economic risk factors identified from health-related data, collecting data related to the intervention plan from the users, and transmitting the identities and collected data;
a server networked to the health stations for receiving the identities and collected data;
a tracking component coupled to the server for tracking compliance with the intervention plan; and
a processing component coupled to the server for calculating a credit to the users for compliance with the intervention plan.

19. The system of claim 18, wherein the health-related data comprises health risk appraisal data.

20. The system of claim 18, wherein the health-related data comprises biometric data.

21. The system of claim 18, wherein the health-related data comprises utilization data.

22. The system of claim 18, wherein the server component delivers one or more intervention activities that reduce the economic risk factors through one of the health stations.

23. The system of claim 22, wherein the credit is based at least in part on participating in the intervention activities.

24. The system of claim 22, wherein the tracking component further tracks the users' participation in the intervention activities by verifying the identities of the users before beginning the intervention activities and after completion of the intervention activities; and wherein the credit is based at least in part on participating in the intervention activities.

25. The system of claim 22, wherein the tracking component further tracks the users' participation in the intervention activities by verifying the identities of the users before beginning the intervention activities and after completion of the intervention activities; and wherein the credit is derived from a merit point system based at least in part on participating in the intervention activities.

26. A system for managing health risks, the method comprising:
means for identifying one or more relevant economic risk factors from health-related data collected from a person;
means for providing an intervention plan to the person based on the relevant economic risk factors;
means for authenticating an identity collected from a person at a remote location;
means for exchanging data related to the intervention plan with the person at the remote location; and
means for providing an incentive to the person for complying with the intervention plan.
